(19) United States
(12) Patent Application Publication
(10) Pub. No.: US 2011/0144527 A1
(43) Pub. Date: Jun. 16, 2011

(54) Artificial Airway with Integrated Core Temperature Monitor

(76) Inventors: KongYuan HE, Encino, CA (US); Fanping Wang, Encino, CA (US)

(21) Appl. No.: 12/635,128

(22) Filed: Dec. 10, 2009

(51) Int. Cl. A61B 5/01; A61M 16/04

(52) U.S. Cl. 600/549; 128/207.15

(57) ABSTRACT

This is a device for use in clinical, surgical intro- and post-operative patient core temperature monitoring, which utilizes an artificial airway to integrate a temperature probe, removing the need for an external cord for connecting to an external display unit, or the traditional way of an esophageal tube that necessitates more medical procedures. Consequently, for patients under general anesthesia with ET Tube or LMA, accurate monitoring of core temperature becomes feasible. As a result, the overall cost for surgical operation, including that of anesthesia, is reduced and the with reduced risk of complication associated with extra oral procedural for inserting an esophageal tube to measure temperature.

### FIELD AND BACKGROUND OF THE INVENTION

The present invention relates generally to clinical, surgical and intro-operative monitoring of patients' temperature and especially who undergo general anesthesia. Particularly, the present invention provides a means for accurate measurement of core body temperatures of patients, both during surgical operation and the post-surgery recovery, and thus provides better monitoring of patients' physical condition.

Traditionally, clinical and surgical temperature measurement and monitoring is done by surface measurement or a specially designed intrusive means, usually some type of PA (Pulmonary Artery) catheter, esophageal, rectal or urinal intubation, to gain access to patients' inner body chamber to get temperature reading. The most common one is to measure the esophageal temperature by placing probe of roughly 45 cm into that part of the esophagus to measure core temperature.

Normally, an anesthesiologist places an esophageal probe after anesthesia induction. The esophageal probe is connected to a central monitor through a special cord, and causes the temperature reading to be displayed on a screen of the external monitor.

The disadvantage of this traditional method includes:

1. The procedure of placing/inserting the esophageal probe into a patient's body takes additional time and effort. Sometimes it can be difficult and does add potential risk factors to the patients.

2. The esophageal probe cannot be placed in patients who relied upon the use of LMA (Laryngeal Mask Airway). In the typical practice were LMA is used, doctors need to apply a skin-sensing strip or other means of temperature monitoring to patients for checking on patients' temperature. Apparently, the skin temperature does not give accurate patient information related to the patients' core body temperature, in response to any medical treatment, surgery, administration of drugs, or the progression of post-operation recovery. If other means are used, there is concern for the accuracy as well.

3. Placing esophageal probe may cause sore throat or even oral injury.

4. It takes up additional oral space, which could be inconvenient for certain surgeries.

5. An external cord going from the patient to an external monitor is needed, which causes inconvenience to medical persons, including anesthesiologist, and thus also increases risk factors for tripping people around the patient, and is a potential threat to patient, consequently.

6. The esophageal temperature probes used associated with this traditional method is a bio-waste.

All these disadvantages prompted the improvements proposed by present invention.

### SUMMARY OF PRESENT INVENTION

An advantage of present invention is that the measurement of core body temperature will be taken from upper airway, instead of the esophagus. The measuring device is the adapted LMA or endo-tracheal tube described herein and thus is a lot simpler and safer to use for surgical, intro-operative or post-operative purposes.

By obtaining vital signs, such as core temperature, from the patient's airway, the process is made simpler helps to reduce costs of extra procedures for monitoring temperature.

The important feature is to embed or integrate a temperature probe to the LMA and/or endo-tracheal tube, removing the need to insert the esophageal tube/probe. Additionally, precise core temperature can be monitored even when the patients are under general anesthesiology with LMA.

Moreover, by having a 2-unit construction, where only the in-body portion needs to be sterilized and the outside portion will be connected at time when it's ready for use, the bio-waste is reduced to only the in-body portion.

### DESCRIPTION OF THE DRAWINGS

The accompanying drawings, which are incorporated in and constitute a part of this specification, illustrate the preferred embodiments of the invention and together with the description, serve to explain the principles of the invention, but are not intended to limit the scope of present invention to the extent present invention is applicable.

FIG. 1 shows the core temperature monitor's overall structure.

FIG. 2 shows a simplified view of first embodiment of present invention.

FIG. 3 shows a cut-out structural view of present invention where the in-body portion temperature-sensing monitor can be optionally connected to an external monitor device, as compared to the integrated display unit (the second portion, see details later.)

FIGS. 4a and 4b show the two-unit construction of present invention.

FIG. 5 shows a second embodiment of present invention built to a LMA.

### DETAILED DESCRIPTION OF PREFERRED EMBODIMENT

The device of present invention is made up of two major portions, the first portion is a in-body portion, having a thermo-couple with conductive signal wires embedded inside the tubing wall of the LMA or endo-tracheal tube, with a display holder. The second portion contains a temperature display unit with digital readout, a printed circuit board and a battery (inside a battery compartment). These two portions can stay separated until ready for use.

For sterilization, only the first portion needs to be sterilized, since only this in-body portion will be place inside a patient's body.

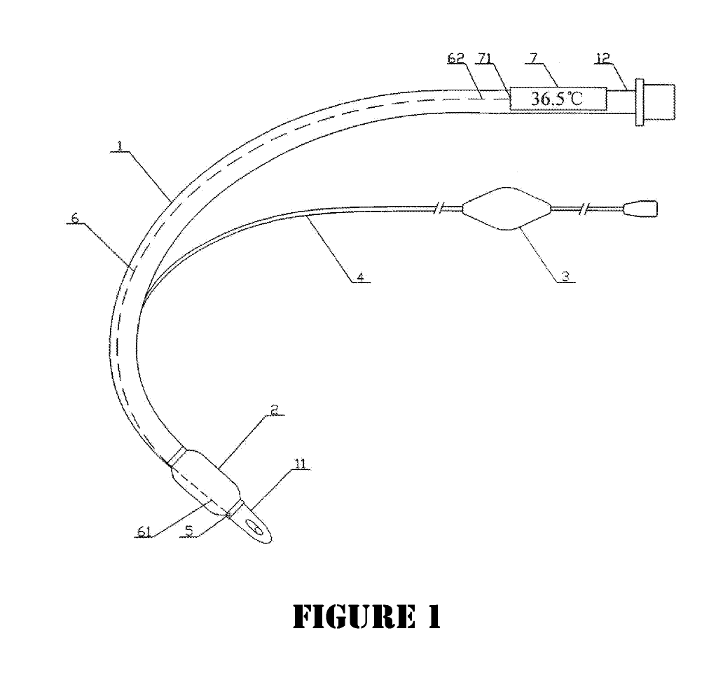

Referring to FIG. 1, where an air tube 1 has an inside end 11 and an outside end 12. A temperature probe 5 is located near the tip of said inside end 11.

A temperature display unit 7 is made up on a digital display unit, a battery compartment (for receiving a suitable battery) and a switch with associated printed circuit board.

The term “inside” and “outside” is used in reference to the fact that the device of present invention's “inside” end will be placed inside a patient's body, that is, into the upper airway of a patient.

An air sac 2 is formed next to the temperature probe 5 near the inside end 11.

An air pump 3 is attached, via a pumping tube 4, to the air sac 2, so that the air tube 1 looks like it has something “forking” out, with one fork going to the air pump 3 and the other fork going to the temperature display unit 7.

A signal wire 6 is placed inside the air tube 1, connecting the temperature probe 5 to the connecting socket 8, which then allows connection to either a display unit 7 or to an external monitor 9.

Note that the signal wire 6 may be a pair of “wires”, although it is used in its singular form herein. The implementation of a signal wire 6 is known art and need no disclosure by present application, and does not constitute any novelty part of present application, except to the extent that it forms part of the complete disclosure in combination with other parts of present invention.

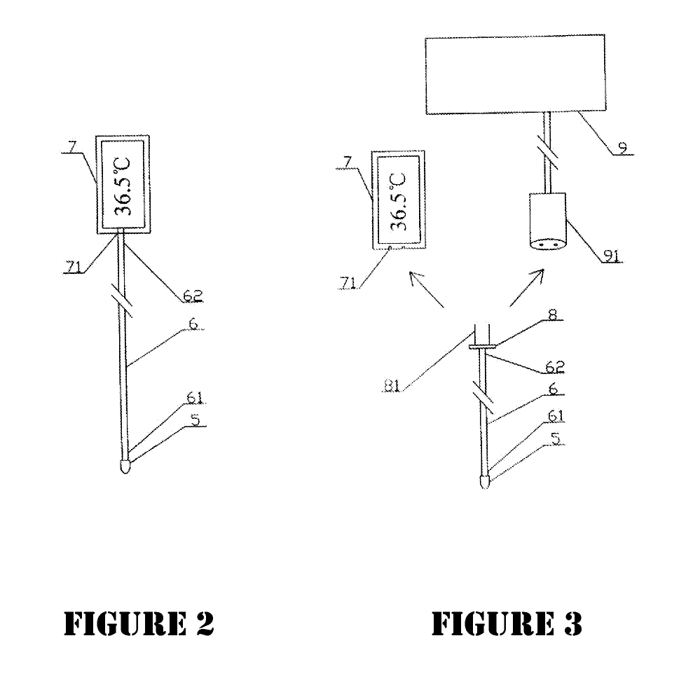

Said signal wire 6 has a probing end 61, which is connected to the temperature probe 5. Said signal wire has an external end 62, as shown in FIG. 2 and FIG. 3, that can be connected to a connecting socket 8, whose connector 8 then in turns connects to the temperature display unit 7, or to an external multi-function monitoring device 9 via an additional cord, having an interface 91.

The placement of temperature probe 5 can be put either on the wall of the air tube 1, or on the wall of the air sac 2.

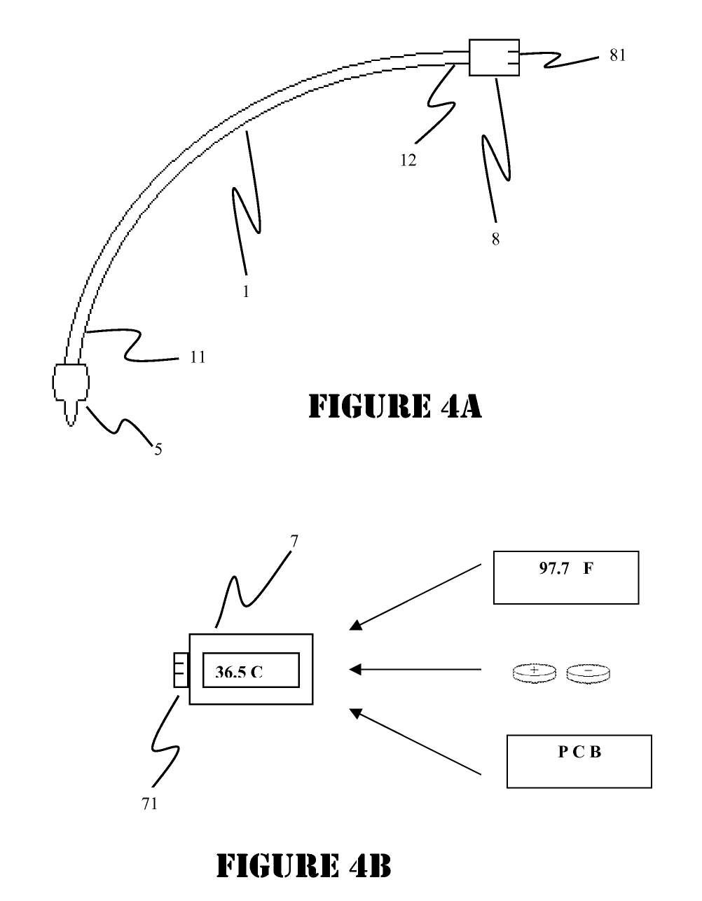

The in-body portion of present invention consists of the portion of the air tube 1 from the inside end 11 to the outside end 62, which ends with a connecting socket 8, containing a connector 81. See FIG. 4a and FIG. 4b for such suggested 2-unit construction.

The outside-body portion of present invention has a connector 7 for sending signal to display unit 7, as shown in FIG. 4b.

By the 2-unit construction of present invention, the external end 62 of said air tube 1 also allows connection to the interface 91 of the external monitoring device 9 via the additional cord.

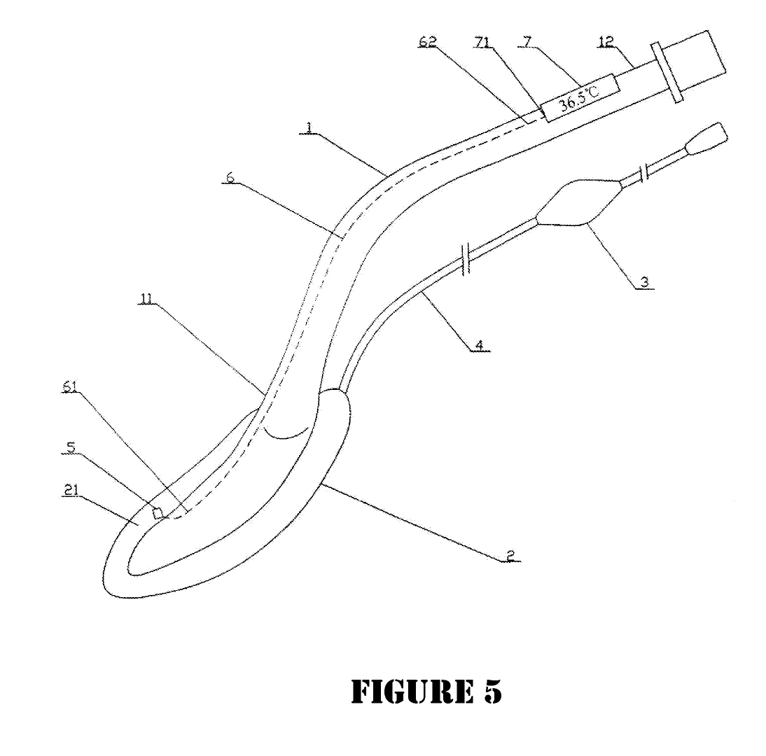

FIG. 5 shows a second embodiment of present invention is specifically built to a LMA, where the temperature probe 5 is placed on the sac wall 21 of the air sac 2. Other features for constructing the integrated temperature monitor device is similar to the air tube 1 as discussed earlier.

A battery compartment can be built near the outside end 12 of said air tube 1, as well as a switch in association with a printed circuit board, so that the device can be turned on/off by the switch. This can be done on the outside-body portion, as suggested in FIG. 4b.

As such, this type of integrated temperature probe with display unit can be applied to other implementations of the same nature which should be considered within the scope of present invention.

What is claimed is:

1. An artificial airway with core temperature monitor, comprising:
An in-body portion having an air tube containing an inside end and an outside end;
A temperature probe located near the tip of said inside end of said air tube;
A signal wire inside said air tube, connecting said temperature probe to a connecting socket near said outside end;
An outside body portion having a temperature display unit having a connector for attaching to the connecting socket on said air tube; and,
An air sac located near the inside end of said air tube and an air pump connect to said air sac, whereby said air pump can be operated to inflate said air sac by a pumping tube connected to said air sac.

2. The device of claim 1, wherein said signal wire further having a probing end connected to said temperature display unit and an external end that contains a wire connecting socket with a connector for connecting to either the connector of said temperature display unit or an interface for an external display.

3. The device of claim 1, wherein said temperature probe is embedded in the inside wall of said air tube.

4. The device of claim 1, wherein said temperature probe is embedded on the sac wall of said air sac.

5. The device of claim 1, wherein said temperature display unit further having a digital display readout, battery compartment and a switch in association with a printed circuit board.

6. An artificial airway with core temperature monitor, comprising:
An in-body portion having an air tube containing an inside end and an outside end;
A temperature probe located near the tip of said inside end of said air tube;
An air sac located near the inside end of said air tube and an air pump connected to said air sac, whereby said air pump can be operated to inflate said air sac by a pumping tube connected to said air sac; and,
A signal wire inside said air tube, connecting said temperature probe to a connecting socket near said outside end, whereby said connecting socket allows the temperature sensing and reading to be sent to an external monitor by an external cord.
